(19) United States
(12) Patent Application Publication
(10) Pub. No.: US 2003/0139349 A1
(43) Pub. Date: Jul. 24, 2003

(54) Benzophenone alpha-d-glycopyranosides, preparation and therapeutic use

(76) Inventors: Luc Lebreton, Dijon (FR); Christiane Legendre, Velars-sur-Ouche (FR); Soth Samreth, Daix (FR)

Correspondence Address: MERCHANT & GOULD PC, P.O. BOX 2903, MINNEAPOLIS, MN, 55402-0903, US

(21) Appl. No.: 10/168,210

(22) PCT Filed: Dec. 6, 2000
(86) PCT No.: PCT/FR00/03420

(51) Int. Cl. A61K 31/704; C07H 5/06

(52) U.S. Cl. 514/25; 536/17.9; 536/18.7

(57) ABSTRACT

The invention concerns (i) [4-(4-cyanobenzyl)phenyl]α-D-glycopyranosides of formula (I) wherein: the group α-D-glycopyranosyl R represents a α-D-glycopyranosyl, α-D-galactopyranosyl. α-D-mannopyranosyl, α-D-arabinopyranosyl, α-D-lyxopyranosyl, or α-D-ribopyranosyl group: (ii) their esters resulting from the esterification of at least a OH function of each pyranosyl group with a C₂-C₄ alkanoic or a cycloalkanoic acid, as novel industrial products. Said novel [4-(4-cyanobenzyl)phenyl]α-D-glycopyranosides are useful in therapy for fighting against atheromatous plaque.

### FIELD OF THE INVENTION

The present invention relates, by way of novel industrial products, to 4-cyano-4′-hydroxybenzophenone derivatives of formula I below, which are benzophenone α-D-glycopyranosides. It further relates to the process for their preparation and to their use in therapeutics, especially in the form of compositions in which they are present as active principles.

### PRIOR ART

EP-A-0051023 has disclosed compounds which contain a hydroxybenzo-phenone residue substituted by a β-D-xylosyl group and which have valuable pharmacological activity for the treatment or prevention of venous thrombosis.

Also, EP-A-0133103 has disclosed derivatives of the benzylphenyl β-D-xyloside type which possess hypocholesterolemic and hypolipidemic properties. It is also known that derivatives in which the β-D-xylosyl radical has been replaced with a β-D-thioxylosyl radical have been described in EP-A-0365397 and EP-A-0290321, said compounds being useful on account of their antithrombotic activity.

Finally, the article by F. BELLAMY et al., J. Med. Chem., 1993, 36 (no. 7), pages 898-903, has disclosed compounds derived from benzophenone substituted by glycosyl groups, among which only the derivatives of the β configuration have antithrombotic activity. A study of these products demonstrated that these compounds, particularly those containing a β-D-xylosyl group, were good substrates for galactosyltransferase I and, consequently, were capable of initiating the synthesis of glycosaminoglycans (GAGs). This mode of action, obtained after oral administration of the product, is very probably responsible for the antithrombotic activity, and only those derivatives in which the D-xylose is of the β configuration exhibit activity in this therapeutic field. There is therefore a correlation between the action on GAG synthesis and the antithrombotic activity which meant that the compounds other than those derived from β-D-xylose were of no value in this therapeutic field.

### OBJECT OF THE INVENTION

According to the invention, it is proposed to provide a novel technical solution for obtaining novel products of therapeutic value in respect of arterial atheromatous plaque, either for treating said plaque or for preventing its appearance.

### SUBJECT OF THE INVENTION

According to the novel technical solution of the invention, [4-(4-cyanobenzoyl)phenyl]α-D-glycopyranoside compounds are used which, surprisingly, in the light of the publications cited above, exhibit activity in the prevention or regression of arterial atheromatous plaque.

The novel products according to the invention are selected from the group consisting of:

(i) the [4-(4-cyanobenzoyl)phenyl]α-D-glycopyranosides of formula I:

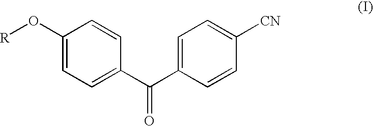

in which the α-D-glycopyranosyl group R is an α-D-glucopyranosyl, α-D-galactopyranosyl, α-D-mannopyranosyl, α-D-arabinopyranosyl, α-D-lyxopyranosyl or α-D-ribopyranosyl group; and

(ii) their esters resulting from the esterification of at least one OH group on each glycopyranosyl group by a C₂-C₄ alkanoic or cycloalkanoic acid.

According to a second feature of the invention, a process is proposed for the preparation of the compounds of formula I above and their esters.

According to yet a third feature of the invention, a therapeutic composition is provided which contains, in association with a physiologically acceptable excipient, a therapeutically effective amount of at least one compound of formula I or one of its esters.

According to another feature of the invention, it is also recommended to use a compound of formula I or one of its esters as an active principle for the preparation of a drug to be used in therapeutics for combating atheromatous plaque, particularly for its prevention or treatment.

### DETAILED DESCRIPTION

The novel compounds according to the invention comprise the products of formula I and their esters; they are pyranoside derivatives of 4-cyano-4′-hydroxy-benzophenone [or 4-(4-hydroxybenzoyl)benzonitrile]. The preferred products, in which the glycoside radical is in the pyranose form, have the formulae below, which are given according to the structure of the glycopyranosyl group R of the α-D configuration:

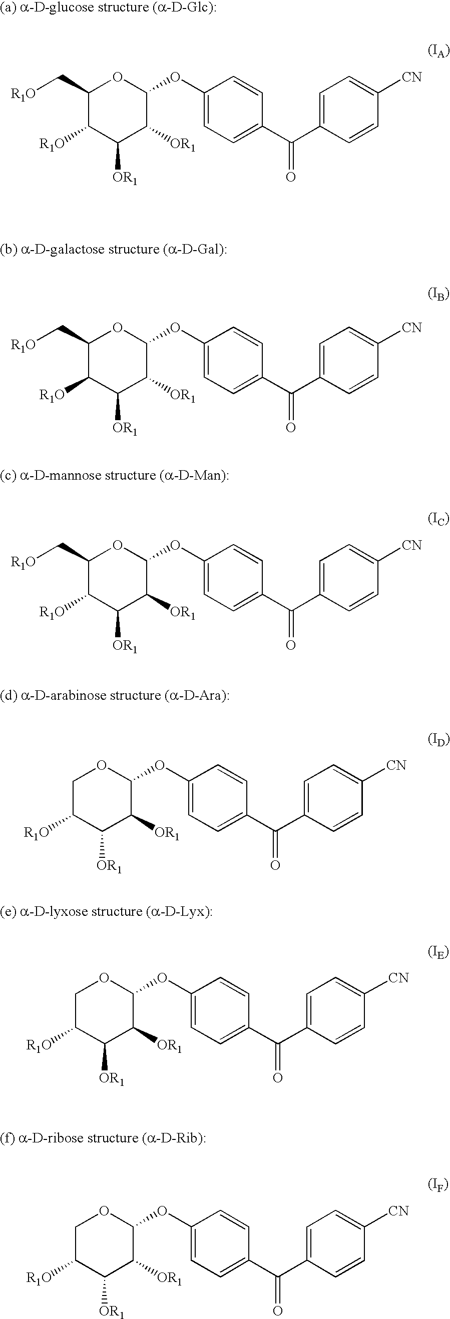

In these formulae, R₁ is a hydrogen atom or a group COR₂, R₂ being a C₁-C₃ alkyl group selected from methyl, ethyl, propyl, isopropyl and cyclopropyl groups.

The process for the preparation of a compound of formula I or one of its esters according to the invention comprises:

(1°) reacting a peracetylated pentose or hexose of the pyranosyl structure of formula II:

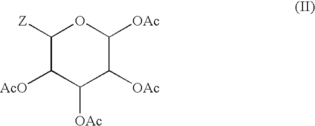

in which Z is H or CH₂OAc,

selected from the group consisting of 1,2,3,4,6-pentaacetyl-D-glucose, 1,2,3,4,6-pentaacetyl-D-galactose, 1,2,3,4,6-pentaacetyl-D-mannose, 1,2,3,4-tetraacetyl-D-arabinose, 1,2,3,4-tetraacetyl-D-lyxose and 1,2,3,4-tetraacetyl-D-ribose,

with 4-(4-hydroxybenzoyl)benzonitrile of formula III:

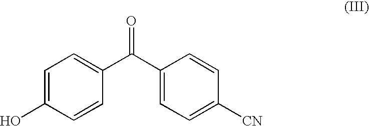

to give, after purification, the corresponding oside compound of formula IV:

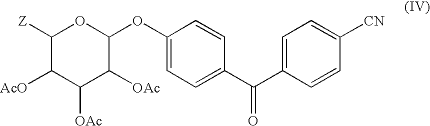

in which Z is defined as indicated above; and

(2°) if necessary, carrying out a displacement reaction on the acetyl groups of the resulting oside compound of formula IV in order to replace them with hydrogen atoms to give the corresponding compound of formula I in which R₁ is H, it being possible for the other esters (in which R₁ is other than Ac) to be obtained by esterifying the compound of formula I in which R₁ is H with a C₃-C₄ acid.

Advantageously, the reaction II+III of step (1°) is carried out in an organic solvent (especially dichloromethane), in the presence of a Lewis acid (for example tin tetrachloride), at a temperature between 25° C. and the boiling point of the solvent, for 10 to 30 hours.

In step (2°), the replacement of the Ac groups with hydrogen atoms is advantageously performed as follows. The compound of formula IV is reacted with NH₃ in solution in an anhydrous alcohol (especially methanol) in order to displace the Ac groups and replace them with H.

In a variant, the reaction II+III→IV of step (1°) can be replaced with the reaction V+III→IV, where V is a corresponding peracetylated halogenopentose or halogenohexose. Under these circumstances, step (1°) becomes step (1′) below, namely:

(1′) reacting a peracetylated halogenopentose or halogenohexose of the pyranosyl structure of formula V:

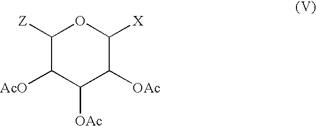

in which X is a halogen atom (i.e. F, Cl, Br or I, the preferred halogen atom being Br) and Z is H or CH₂OAc,

selected from the group consisting of 1-bromo-2,3,4,6-tetraacetyl-D-glucose, 1-bromo-2,3,4,6-tetraacetyl-D-galactose, 1-bromo-2,3,4,6-tetraacetyl-D-mannose, 1-bromo-2,3,4-triacetyl-D-arabinose, 1-bromo-2,3,4-triacetyl-D-lyxose and 1-bromo-2,3,4-triacetyl-D-ribose,

with 4-(4-hydroxybenzoyl)benzonitrile of formula III:

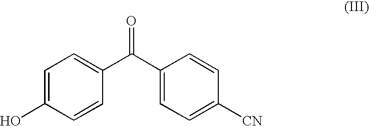

to give, after purification, the corresponding oside compound of formula IV:

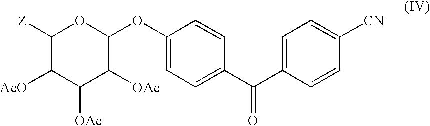

in which Z is defined as indicated above.

Advantageously, the reaction V+III→IV is carried out in an anhydrous solvent such as dichloromethane, 1,2-dichloroethane or acetonitrile, in the presence of a coupling agent such as silver trifluoromethanesulfonate or silver oxide, at a temperature of the order of −10 to +10° C., for 5 to 40 hours.

The reactions II+III→IV and V+III→IV are applicable to the preparation of all the compounds of formula IV according to the invention.

Other advantages and characteristics of the invention will be understood more clearly from the following Preparatory Examples and pharmacological tests. Of course, these details as a whole do not imply a limitation but are provided by way of illustration.

### EXAMPLE I (FORMULA I_{A}, R₁=COCH₃)

[4-(4-Cyanobenzoyl)phenyl]2,3,4,6-tetra-O-acetyl-α-D-glucopyranoside

A suspension of 12.17 g (31.10⁻³ mol) of 1,2,3,4,6-penta-O-acetyl-β-D-glucose and 10.36 g (46.10⁻³ mol) of 4-(4-hydroxybenzoyl)benzonitrile in 500 ml of dichloromethane is prepared and 8.5 ml (72.6.10⁻³ mol) of anhydrous tin tetrachloride are added gradually at 0° C., with stirring. After stirring for 20 hours at room temperature, a further 4.25 ml (36.3.10⁻³ mol) of anhydrous tin tetrachloride are added and the reaction mixture is refluxed gently for 24 hours. After cooling, the reaction medium is poured onto ice. The organic phase is separated off, then washed with water, extracted with 1 N sodium hydroxide solution, then washed with water until the washings are neutral, and dried over magnesium sulfate. After concentration under reduced pressure, the residue is purified by chromatography on silica gel using a toluene/ethyl acetate mixture (8/2; v/v) as the eluent to give 918 mg of the expected product in the form of an amorphous white solid (yield=5.3%).

M.p.=63° C.

[α]_{D}²⁹=+83.2° (c=0.25; DMSO)

### EXAMPLE 2 (FORMULA I_{A}, R₁=H)

[4-(4-Cyanobenzoyl)phenyl]α-D-glucopyranoside

A solution of 575 mg (1.06.10⁻³ mol) of the compound obtained according to Example 1 in 50 ml of methanol is prepared and 5.6 ml of a saturated solution of ammonia in methanol are added at 0° C., with stirring. The reaction mixture is subsequently stirred for 6 hours at room temperature and the solvent is then driven off under reduced pressure. The crude product obtained is purified by chromatography on silica gel using a dichloromethane/methanol mixture (9/1; v/v) as the eluent to give 137 mg of the expected product in the form of a fine white solid (yield=34%).

M.p.=130° C.

[α]_{D}²³=+145° (c=0.33; DMSO)

### EXAMPLE 3 (FORMULA I_{C}, R₁=COCH₃)

[4-(4-Cyanobenzoyl)phenyl]2,3,4,6-tetra-O-acetyl-α-D-mannopyranoside

A solution of 9.9 g (144.10⁻³ mol) of 4-(4-hydroxybenzoyl)benzonitrile and 26 g (63.10⁻³ mol) of 2,3,4,6-tetra-O-acetyl-α-D-mannopyranosyl bromide in 300 ml of 1,2-dichloroethane is prepared in the presence of about 4 g of a molecular sieve. The mixture is cooled to −20° C. and 34 g (132.10⁻³ mol) of silver trifluoromethanesulfonate are added at this temperature. The reaction mixture is stirred for 24 hours at 0° C. and then filtered to remove the solid particles. The organic phase is washed with dilute hydrochloric acid solution, then with water, then with dilute sodium hydroxide solution and finally with water. After drying over magnesium sulfate, the solution is concentrated under reduced pressure and the crude product obtained is purified by chromatography on silica gel using a toluene/ethyl acetate mixture as the eluent to give 19 g of the expected product in the form of a pale yellow solid (yield=77%).

M.p.=60° C.

[α]_{D}²⁷=+64° (c=0.62; DMSO)

### EXAMPLE 4 (FORMULA I_{C}, R₁=H)

[4-(4-Cyanobenzoyl)phenyl]α-D-mannopyranoside

16.7 g (30.10⁻³ mol) of the product obtained according to Example 3 are dissolved in 50 ml of methanol, and 100 ml of a saturated solution of ammonia in methanol are added at 0° C. The reaction mixture is stirred for 6 hours at 0-10° C. and then concentrated under reduced pressure. The crude product is purified by chromatography on silica gel using a dichloromethane/methanol mixture (15/1; v/v) as the eluent. The pure product fraction is crystallized from acetone to give 7.9 g of the expected product in the form of fine light beige crystals (yield=68%).

M.p.=145° C.

[α]_{D}²⁷=+102° (c=0.17; DMSO)

### EXAMPLE 5 (FORMULA I_{B}, R₁=COCH₃)

[4-(4-Cyanobenzoyl)phenyl]2,3,4,6-tetra-O-acetyl-α-D-galactopyranoside

The expected product is obtained in the form of an amorphous solid with a yield of 4% by following a procedure analogous to Example 1 and starting from 1,2,3,4,6-penta-O-acetyl-D-galactose.

M.p.=64-66° C.

[α]_{D}²⁹=+156° (c=0.26; DMSO)

### EXAMPLE 6 (FORMULA I_{B}, R₁=H)

[4-(4-Cyanobenzoyl)phenyl]α-D-galactopyranoside

The expected product is obtained in the form of a white solid with a yield of 90% by following a procedure analogous to Example 2 and starting from the compound obtained according to Example 5.

M.p.=240° C.

[α]_{D}²⁹=+173° (c=0.25; DMSO)

### EXAMPLE 7 (FORMULA I_{D}, R₁=COCH₃)

[4-(4-Cyanobenzoyl)phenyl]2,3,4-tri-O-acetyl-α-D-arabinopyranoside

The expected product is obtained in the form of a yellow oil with a yield of 15% by following a procedure analogous to Example 3 and starting from 2,3,4-tri-O-acetyl-D-arabinopyranosyl bromide.

[α]_{D}²⁴=−10.7° (c=0.38; CH₂Cl₂)

### EXAMPLE 8 (FORMULA I_{D}, R₁=H)

[4-(4-Cyanobenzoyl)phenyl]α-D-arabinopyranoside

The expected product is obtained in the form of a fine beige solid with a yield of 75% by following a procedure analogous to Example 4 and starting from the compound obtained according to Example 7.

M.p.=160° C.

[α]_{D}²⁶=−66° (c=0.32; DMSO)

### EXAMPLE 9 (FORMULA I_{E}, R₁=COCH₃)

[4-(4-Cyanobenzoyl)phenyl]2,3,4tri-O-acetyl-α-D-lyxopyranoside

The expected product is obtained in the form of an oil with a yield of 60% by following a procedure analogous to Example 1 and starting from 1,2,3,4-tetra-O-acetyl-D-lyxopyranose.

[α]_{D}²⁴=+40.3° (c=0.67; CH₂Cl₂)

### EXAMPLE 10 (FORMULA I_{E}, R₁=H)

[4-(4-Cyanobenzoyl)phenyl]α-D-lyxopyranoside

The expected product is obtained in the form of a white solid with a yield of 90% by following a procedure analogous to Example 2 and starting from the compound obtained according to Example 9.

M.p.=173° C.

[α]_{D}²⁸=+125° (c=0.175; DMSO)

### EXAMPLE 11 (FORMULA I_{F}, R₁=H)

[4-(4-Cyanobenzoyl)phenyl]α-D-ribopyranoside

[4-(4-Cyanobenzoyl)phenyl]2,3,4-tri-O-acetyl-α-D-ribopyranoside is obtained in the form of a yellow solid by following a procedure analogous to Example 1 and starting from 1,2,3,4-tetra-O-acetylribopyranose. It is treated with a solution of ammonia in methanol according to the protocol described in Example 2 to give the expected product in the form of a white powder with an overall yield of 6%.

M.p.=164° C.

[α]_{D}²⁶=+77.7° (c=0.21; DMSO)

The antiatheromatous activity of the compounds according to the invention was evaluated as a function of their ability to lower the serum cholesterol level in mice subjected to a fatty diet. Several publications have in fact demonstrated a close correlation between an excess of lipids and a marked increase in the risk of atheroma (cf. Lancet 1996, 348, pages 1339-1342; Lancet 1990, 335, pages 1233-1235). This correlation affords a test which is more rapid than direct experiments on the atheromatous plaque, which require a lengthy treatment of the animals and an expensive histological study of the walls of the aortic arch.

The test used consists in administering a single dose of the compound to female mice of the C57BL/6J strain. The protocol is as follows: On the first day (D0), the mice are fasted from 9 am to 5 pm, a blood sample being taken at 2 pm. At 5 pm, a given amount of food (a fatty diet comprising 1.25% of cholesterol and 0.5% of cholic acid) is distributed. On the second day (D1), the food leftovers are weighed at 9 am and the mice are fasted from 9 am to 2 pm. A blood sample is taken at 2 pm. For the treated groups of mice, the compound is administered at 9 am on the second day (D1) by tubage in the form of a suspension in a 3% aqueous solution of gum. The control groups receive only the aqueous gum.

The compounds were tested at a dose of 100 mg/kg. The total serum cholesterol is assayed and the results are expressed as the percentage inhibition of the increase in cholesterolemia compared with the control group. The results obtained are given in the “Activity” column of Table I. It may furthermore be noted that analysis of the cholesterol content of the different classes of serum lipoproteins shows a favorable effect of the product on the ratio HDL cholesterol/total cholesterol.

It was also demonstrated that the compounds of formula I according to the invention do not induce GAG synthesis.

The products of formula I and their esters according to the invention can preferably be administered orally in the form of tablets or gelatin capsules each containing 20 to 500 mg of a compound of formula I or one of its esters as the active principle, in association with excipients. The dosage will be about 1 to 4 units per day. The products according to the invention are advantageously prescribed for atheromatous plaque and particularly for preventing or treating the risk of atheroma.

TABLE 1

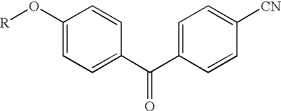

| Ex. | R | R₁ | Activity (%) |
|---|---|---|---|
| 1 | α-D-Glc | COCH₃ | −27 |
| 2 | α-D-Glc | H | −23 |
| 3 | α-D-Man | COCH₃ | −10 |
| 4 | α-D-Man | H | −38 |
| 5 | α-D-Gala | COCH₃ | −32 |
| 6 | α-D-Gala | H | −35 |
| 7 | α-D-Ara | COCH₃ | −25 |
| 8 | α-D-Ara | H | −29 |
| 9 | α-D-Lyx | COCH₃ | — |
| 10 | α-D-Lyx | H | −48 |
| 11 | α-D-Rib | H | −26 |

1. An α-D-glycopyranoside compound, characterized in that it is selected from the group consisting of:
(i) the [4-(4-cyanobenzoyl)phenyl]α-D-glycopyranosides of formula I: 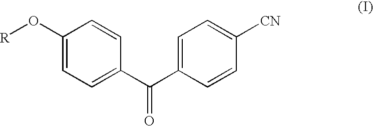
in which the α-D-glycopyranosyl group R is an α-D-glucopyranosyl, α-D-galactopyranosyl, α-D-mannopyranosyl, α-D-arabinopyranosyl, α-D-lyxopyranosyl or α-D-ribopyranosyl group; and
(ii) their esters resulting from the esterification of at least one OH group on each glycopyranosyl group by a C₂-C₄ alkanoic or cycloalkanoic acid.

2. [4-(4-Cyanobenzoyl)phenyl]α-D-glucopyranoside and its peracetylated derivative.

3. [4-(4-Cyanobenzoyl)phenyl]α-D-galactopyranoside and its peracetylated derivative.

4. [4-(4-Cyanobenzoyl)phenyl]α-D-mannopyranoside and its peracetylated derivative.

5. [4-(4-Cyanobenzoyl)phenyl]α-D-arabinopyranoside and its peracetylated derivative.

6. [4-(4-Cyanobenzoyl)phenyl]α-D-lyxopyranoside and its peracetylated derivative.

7. [4-(4-Cyanobenzoyl)phenyl]α-D-ribopyranoside and its peracetylated derivative.

8. A pharmaceutical composition, characterized in that it contains, in association with a physiologically acceptable excipient, a therapeutically effective amount of at least one compound of formula I or one of its esters according to claim 1.

9. The use of a product selected from the group of compounds of formula I and their esters according to claim 1 for the preparation of an antiatheromatous drug to be used in therapeutics for combating atheromatous plaque.

10. A process for the preparation of a [4-(4-cyanobenzoyl)phenyl]α-D-glycopyranoside compound of formula I or its peracetylated derivative according to claim 1, said process being characterized in that it comprises:
(1°) reacting (a) a peracetylated pentose or hexose of the pyranosyl structure of formula II: 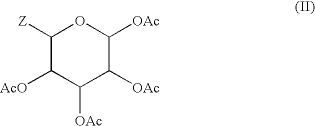
in which Z is H or CH₂OAc,
selected from the group consisting of 1,2,3,4,6-pentaacetyl-D-glucose, 1,2,3,4,6-pentaacetyl-D-galactose, 1,2,3,4,6-pentaacetyl-D-mannose, 1,2,3,4-tetraacetyl-D-arabinose, 1,2,3,4-tetraacetyl-D-lyxose and 1,2,3,4-tetraacetyl-D-ribose,
or (b) a peracetylated halogenopentose or halogenohexose of the pyranosyl structure of formula V: 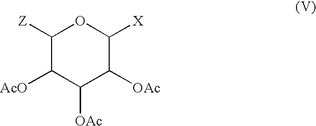
in which X is a halogen atom (i.e. F, Cl, Br or I, the preferred halogen atom being Br) and Z is H or CH₂OAc,
selected from the group consisting of 1-bromo-2,3,4,6-tetraacetyl-D-glucose, 1-bromo-2,3,4,6-tetraacetyl-D-galactose, 1-bromo-2,3,4,6-tetraacetyl-D-mannose, 1-bromo-2,3,4-triacetyl-D-arabinose, 1-bromo-2,3,4-triacetyl-D-lyxose and 1-bromo-2,3,4-triacetyl-D-ribose,
with 4-(4-hydroxybenzoyl)benzonitrile of formula III: 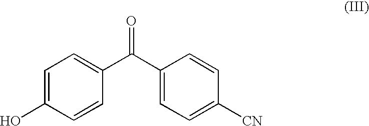
to give, after purification, the corresponding oside compound of formula IV: 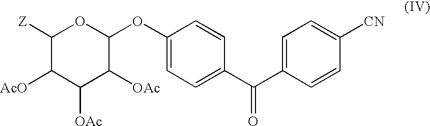
in which Z is defined as indicated above; and
(2°) if necessary, carrying out a displacement reaction on the acetyl groups of the resulting oside compound of formula IV in order to replace them with hydrogen atoms to give the corresponding compound of formula I in which R₁ is H.
